(19) United States
(12) Patent Application Publication
(10) Pub. No.: US 2004/0028640 A1
(43) Pub. Date: Feb. 12, 2004

(54) Cosmetic care or make-up composition for keratin materials comprising an aliphatic ester

(76) Inventors: Pascal Arnaud, Les Roses (FR); Vanina Filippi, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/415,339

(22) PCT Filed: Sep. 20, 2002
(86) PCT No.: PCT/IB02/04224

(30) Foreign Application Priority Data
Sep. 21, 2001 (FR) 0112216

(51) Int. Cl. A61K 7/075; A61K 7/08

(52) U.S. Cl. 424/70.31

(57) ABSTRACT

The invention relates to a cosmetic care or make-up composition for keratin materials comprising a cosmetically acceptable medium containing i) at least one aliphatic ester of an ester resulting from the esterification, by an aliphatic carboxylic acid, of an aliphatic hydroxycarboxylic acid ester and ii) at least one oil of high molar mass ranging from 650 to 10000 g/mol. This composition possesses remarkable cosmetic properties and confers, in particular, on the make-up or care properties of gloss, of slipperiness during application, and of comfort. The invention also relates to the use of the said aliphatic ester of an ester and of the said oil having a molar mass ranging from 650 to 10000 g/mol, in a physiologically acceptable composition, as agent for conferring on the said composition properties of slipperiness, of nonstickiness, of gloss and/or of comfort.

The present invention relates to a cosmetic make-up or care composition for the skin, including the scalp, both of the human face and body, of the lips or of the superficial body growths of human beings, such as the hair, the eyelashes, the eyebrows or the nails, comprising a cosmetically acceptable medium containing an aliphatic ester of a particular ester. This composition possesses remarkable cosmetic properties and confers, in particular, on the make-up or care properties of gloss, of slipperiness during application, and of comfort.

The composition of the invention may, in particular, constitute a make-up product for the body, the lips or the superficial body growths of human beings hating in particular properties of nontherapeutic care and/or treatment. It constitutes in particular a lipstick or a lip gloss, a blusher or an eyeshadow, a tattoo product, a mascara, an eyeliner, a nail varnish, an artificial tanning product for the skin, a dye or care product for the heir.

Numerous cosmetic compositions exist for which the properties of gloss and comfort of the deposited film, after application to the keratin materials (skin, lips, superficial body growths), are desirable. There may be mentioned, for example, lipsticks, nail varnishes or certain hair products.

In this perspective, the formulator has available several types of raw materials, and in particular lanolins used in combination with so-called “glossy” oils such as oily polymers such as polybutenes which have a high viscosity (typically greater than 2819 Pa.s at 23° C. measured with a Brookfield Rv viscometer equipped with a No. 1 needle revolving at 0.5 rpm), fatty acid or alcohol esters in which the number of carbons is high. (typically greater than 16), or certain vegetable oils.

The lanolins have, nevertheless, the disadvantage of being sensitive to heat and to ultraviolet radiation, and tend to become oxidized with an emission of an unpleasant odour, which limits their use in cosmetic compositions.

Furthermore, when the lanolins are combined with oils commonly used in the cosmetic field, the compositions obtained have problems of stickiness which are all the more pronounced if the oil used possesses a high viscosity.

Moreover, cosmeticians are currently seeking to replace the raw materials of animal origin, which is the case for lanolin, with components of non-animal origin, in cosmetic formulations.

The subject of the invention is precisely a cosmetic care or make-up composition for keratin materials, and in particular for the skin and/or the lips and/or the superficial body growths, which makes it possible to overcome the drawbacks cited above.

The applicant has found, surprisingly, that the use i) of at least one (that is to say one or more), aliphatic ester of an ester resulting from the partial or total esterification, with an aliphatic carboxylic acid, of an aliphatic hydroxycarboxylic acid ester, combined with ii) at least one (namely one or more) oil having a molar mass ranging from 650 to 10000 g/mol made it possible to obtain a glossy composition having good properties, upon application and over time, of spreading, of slipperiness, of nonstickiness and of comfort (non-tightness, non-drying).

The properties of gloss and of nonstickiness are in particular remarkable and even superior to those obtained with the lanolins commercially used in the cosmetic field.

The documents KR 2001-047704 and JP 2000-086452 describe cosmetic compositions containing an ester of castor oil, but do not teach that its combination with an oil having a molecular mass of between 650 and 10000 g/mol makes it possible to obtain a glossy product which is comfortable and slippery during application.

Application EP 1 112 734 gives examples of glossy, transfer-free cosmetic compositions which contain an oil of high molecular mass, such as polybutene, a nonvolatile silicone and a volatile solvent, such as isododecane.

In the remainder of the text, the oil having a molecular mass ranging from 650 to 10000 g/mol will be called an oil of high molar mass.

The subject of the invention-is therefore a cosmetic care or make-up composition for keratin materials comprising a cosmetically acceptable medium containing i) at least one aliphatic ester of an ester resulting from the esterification, by an aliphatic carboxylic acid, of an aliphatic hydroxycarboxylic acid ester and ii) at least one oil of high molar mass ranging from 650 to 10000 g/mol.

The subject of the invention is also a cosmetic method for conferring on a cosmetic composition film properties of slipperiness, of nonstickiness, of gloss and/or of comfort, consisting in introducing into the said composition i) at least one aliphatic ester of an ester resulting from the esterification, by an aliphatic carboxylic acid, of an aliphatic hydroxycarboxylic acid ester and ii) at least one oil of high molar mass ranging from 650 to 10000 g/mol.

The subject of the invention is also the use of the combination i) of at least on aliphatic ester of an ester r sulting from the esterification, by an aliphatic carboxylic acid, of an aliphatic hydroxy-carboxylic acid ester and ii) of at least one oil of high molar mass ranging from 650 to 10000 g/mol, in a physiologically acceptable slippery, nonsticky, glossy and/or comfortable composition.

The subject of the invention is finally the use of the combination i) of at least one aliphatic ester of an ester resulting from the esterification, by an aliphatic carboxylic acid, of an aliphatic hydroxy-carboxylic acid ester and ii) of at least one oil of high molar mass ranging from 650 to 10000 g/mol, in a physiologically acceptable composition, as agent for conferring on the said composition properties of slipperiness, of nonstickiness, of gloss and/or of comfort.

Some of the aliphatic esters of esters used in the composition of the invention are known for very specific uses, such as the castor oil esters cited in Application JP-A-08 209 160 by Itoh Oil which describes lubricating compositions for motor cars, boats or aeroplanes, containing these_esters.

There may also be mentioned Application JP-A-05 221 839 by Kanebo in which there is described a hair rinsing composition comprising a combination of quaternary ammonium salts, higher alcohols, acids and an ester which may be an ester of castor oil.

Finally, there is also known Application JP-A-06 055 057 by Itoh Oil whose subject is a transparent gel composition resulting from the combination of a fatty acid of hydrogenated castor oil, of products of addition of ethylene oxide and of castor oil and of esters of hydrogenated castor oil.

However, none of the documents known in the state of the art describes compositions such as those of the present invention, or discloses or suggests the cosmetic properties of the said composition.

The word ester according to the invention means a monoester, a diester, a triester and more generally a polyester.

The aliphatic ester of an ester, combined with the oil of high molar mass ranging from 650 to 10000 g/mol in the composition according to the invention, results from the esterification, by an aliphatic carboxylic acid (which will be subsequently called “second acid”), of an aliphatic hydroxycarboxylic acid ester (which will be subsequently called “first esters”).

The aliphatic carboxylic acid (or second acid) according to the invention advantageously comprises from 4 to 30 and preferably from 8 to 30 carbon atoms, and may b linear or branched.

This aliphatic carboxylic acid may be chosen from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid, docosanoic acid, and mixtures thereof.

Preferably, the aliphatic carboxylic acid (or second acid) is branched. It may be chosen from 2-ethylhexanoic acid, hexyldecanoic acid, isostearic acid, isoarachidic acid, octyldodecanoic acid, and mixtures thereof.

Preferably, the aliphatic carboxylic acid (or second acid) is isostearic acid.

The aliphatic hydroxycarboxylic acid ester (or first ester) is an ester derived from a hydroxylated aliphatic carboxylic acid (which will be called first acids) whose —COOH functional group has been esterified with an alcohol. This (nonesterified) first acid contains in particular from 2 to 40 carbon atoms, preferably from 10 to 34 carbon atoms and even better from 12 to 28 carbon atoms; it contains, in addition, from 1 to 20 hydroxyl groups, preferably from 1 to 10 hydroxyl groups and even better from 1 to 6 hydroxyl groups, which are capable of being subsequently esterified with an aliphatic carboxylic acid (or second acid) in order to obtain the aliphatic ester of an ester used in the present invention.

The alcohol may comprise from 1 to 40 carbon atoms and even better from 3 to 30 carbon atoms. This alcohol may be a monoalcohol or a polyol (comprising at least two —OH groups); preferably, the alcohol is an aliphatic polyol. Advantageously, the polyol is not a monosaccharide.

In the case where the first ester is derived from the reaction of the hydroxylated aliphatic carboxylic acid (or first acid) with a polyol, the said polyol may be partially or totally esterified; advantageously, this first ester is a total ester.

Preferably, the aliphatic hydroxycarboxylic acid ester (or first ester) is a fatty acid ester in which the fatty acid residue contains at least 12 carbon atoms, for example from 12 to 40 carbon atoms, and even better from 12 to 28 carbon atoms.

The aliphatic hydroxycarboxylic acid ester which can be used in the invention may be chosen from:

a) the partial or total esters of saturated linear monohydroxylated aliphatic monocarboxylic acids such as:

the esters of lactic acid such as isostearyl lactate, lactate derived from C₁₂-C₁₃ alcohol, octyldodecyl lactate, oleyl lactate, myristyl lactate;

the esters of 12-hydroxyoctadecanoic (or 12-hydroxystearic) acid such as 2-ethylhexyl hydroxystearate, octyldodecyl hydroxystearate, isostearyl hydroxystearate, isodecyl hydroxy-stearate, glyceryl trihydroxystearate (or hydrogenated castor oil), dipentaerythrityl hexahydroxystearate;

b) the partial or total esters of unsaturated monohydroxylated aliphatic monocarboxylic acids such as the esters of ricinoleic acid (or 12-hydroxy(cis)-9-octadecenoic acid) such as butyl ricinoleate, octyldodecyl ricinoleate, cetyl ricinoleate, glyceryl triricinoleate (castor oil);

c) the partial or total esters of saturated monohydroxylated aliphatic polycarboxylic acids such as diisostearyl malate, triisostearyl citrate, trioctyldodecyl citrate;

d) the partial or total esters of saturated polyhydroxylated aliphatic polycarboxylic acids such as the tartrate derived from the reaction with 2 branched C₁₂-C₁₃ alcohols;

e) the partial or total esters of aliphatic C₂ to C₁₆ polyol which has reacted with a mono- or polyhydroxylated aliphatic mono- or polycarboxylic acid such as in particular triglycerides, pentaerythritol esters, propane trimethylol, propylene glycol, neopentyl glycol, dipentaerythritol, polyglycerol, esters of sorbitol;

and mixtures thereof.

Advantageously when the aliphatic hydroxycarboxylic acid ester (or first ester) results from the esterification of a monohydroxylated aliphatic polycarboxylic acid (or first acid) such as those cited above, no residual COOH group remains which is not involved in an ester bond.

The aliphatic, esters of an ester which are used in the present invention are advantageously compounds which are pasty or viscous at room temperature (25° C.). The expression pasty for the purposes of the invention is understood to mean a compound having a melting point ranging from 25 to 60° C., preferably from 30 to 45° C. and/or a hardness ranging from 0.001 to 0.5 MPa, preferably from 0.005 to 0.4 MPa.

The melting point values correspond to the melting point measured using a differential scanning calorimeter (DSC) such as the calorimeter sold under th name DSC 30 by the company Metler, with a temperature rise of 5 or 10° C. per minute. (The melting point considered is the point corresponding to the temperature of the most endothermic peak of the thermogram).

The hardness is measured according to a method of penetration of a probe into a sample of compound and in particular using a texture analyser (for example TA-XT2i from Rhéo) equipped with a stainless steel cylinder 2 mm in diameter. The measurement of hardness is carried out at 20° C. at the centre of 5 samples. The cylinder is introduced into each sample at a pre-speed of 1 mm/s and then at a measurement speed of 0.1 mm/s, the depth of penetration being 0.3 mm. The hardness value recorded is that of the maximum peak.

Preferably, the aliphatic ester of an ester of the composition according to the invention is chosen from the esters of partial or total esters of aliphatic C₂ to C₁₆ polyols, the said polyols having reacted with an aliphatic hydroxymonocarboxylic acid (first acid).

Advantageously, the aliphatic ester of an ester of the composition according to the invention is chosen from the esters of partial or total esters of aliphatic C₂ to C₁₆ polyols, the said polyols having reacted with a hydroxylated aliphatic fatty acid (or first acid) having a saturated or unsaturated chain, containing at least 12 carbon atoms, for example from 12 to 40 carbon atoms, and even better from 12 to 28 carbon atoms, such as ricinoleic acid, 12-hydroxystearic acid, 14-hydroxyeicosenoic acid.

Preferably, the aliphatic ester of an ester is chosen from the esters of saturated or unsaturated, partial or total esters of aliphatic C₂ to C₁₆ polyols, the said polyols having reacted with ricinoleic acid (first acid).

There may be mentioned in particular, as aliphatic ester of an ester which can be used in the composition according to the invention:

the ester resulting from the esterification reaction of hydrogenated castor oil with isostearic acid in the proportions of 1 to 1 (1/1) or monoisostearate of hydrogenated castor oil,

the ester resulting from the esterification reaction of hydrogenated castor oil with isostearic acid in the proportions of 1 to 2 (1/2) or the diisostearate of hydrogenated castor oil,

the ester resulting from the esterification reaction of hydrogenated castor oil with isostearic acid in the proportions of 1 to 3 (1/3) or triisostearate of hydrogenated castor oil,

and mixtures thereof.

Thes compounds are in a pasty form at room temperature; by way of example, the monoisostearte of hydrogenated castor oil has a melting point, measured by DSC, of 34.6° C. and a hardness of 0.01 MPa.

The quantities of the various ingredients of the composition according to the invention will be given as percentages by weight relative to the total weight of the said composition.

The aliphatic ester of an ester of the composition according to the present invention may represent from 1 to 99% of the total weight of-the composition, preferably from 1 to 75% and even better from 5 to 60%.

The aliphatic ester of an ester of the composition according to the invention may be prepared according to methods known to persons skilled in the art.

The composition according to the invention also contains an oil of high molar mass ranging from 650 to 10000 g/mol. The expression “oil” is understood to mean a nonaqueous compound which is immiscible with water, and which is liquid at room temperature (25° C.) and atmospheric pressure (760 mmHg).

The oil used in the composition according to the present invention has a molar mass ranging from 650 to 10000 g/mol, and is preferably betwe n 750 and 7500 g/mol.

Indeed, oils having an excessively low molar mass, combined with the aliphatic ester of an ester in the composition according to the invention, lead to compositions which are not sufficiently glossy; the oils having an excessively high molar mass give, for their part, compositions which are judged to be too sticky.

Thus, the triglyceride of capric/caprylic acids (such as that marketed or manufactured under the reference ESTOL 3603 NCT OIL by the company Uniqema), which has a molar mass equal to 494 g/mol, leads to compositions which have cosmetic Properties which are not as good as those of the composition of the invention.

The oil having a molar mass ranging from 650 to 10000 g/mol, which can be used in the present invention, may be chosen from:

lipophilic polymers such as:

polybutylenes such as INDOPOL H-100 (having a molar mass or MM=965 g/mol), NDOPOL H-300. (MM=1340 g/mol), INDOPOL H-1500 (MM=2160 g/mol) which are marketed or manufactured by the company AMOCO,

hydrogenated polyisobutylenes such as PANALANE H-300 E which are marketed or manufactured by the company AMOCO (M=1340 g/mol), VISEAL 20000 marketed or manufactured by the company SYNTAL (MM=6000 g/mol), REWOPAL PIB 1000 marketed or manufactured by the company WITCO (MM=1000 g/mol),

polydecenes and hydrogenated polydecenes such as; PURESYN 10 (MM=723 g/mol), PURESYN 150 (MM=9200 g/mol) which are marketed or manufactured by the company MOBIL CHEMICALS,

copolymers of vinypyrrolidone such as: the vinylpyrrolidone/1-hexadecene copolymer, ANTARON V-216, marketed or manufactured by the company IsP (MM=7300 g/mol),

esters such as:

linear fatty acid esters having a total number of carbons ranging from 35 to 70 such as pentaerythrityl tetrapelargonate (MM=697.05 g/mol),

hydroxylated esters such as 2-polyglyceryl triisostearate (MM=965.58 g/mol),

aromatic esters such as tridecyl trimellitate (MM=757.19 g/mol),

C₂₄-C₂₈ branched fatty acid or fatty alcohol esters such as those described in Application EP-A-0 955 039, and in particular triisoarachidyl citrate (MM=1033.76 g/mol), pentaerythrityl tetraisononanoate (MM=697.05 g/mol), glyceryl triisostearate (MM=891.51 g/mol), glyceryl tri(2-decyltetradecanoate) (MM=1143.98 g/mol), pentaerythrityl tetraisostearate (MM=1202.02 g/mol), polyglyceryl-2 tetraisostearate (MM=1232.04 g/mol) or alternatively pentaerythrityl tetra(2-decyltetradecanoate) (MM=1538-66 g/mol),

silicone oils such as phenylated silicone such as BELSIL PDM 1000 from the company WACKER (MM=9000 g/mol),

oils of plant origin such as sesame oil (820.6 g/mol),

and mixtures thereof.

The oil having a molar mass ranging from 650 to 10000 g/mol used in the composition according to the invention may represent from 1 to 99%, preferably from 10 to 80% and even better from 5 to 70% of the total weight of the composition.

Advantageously, the composition of the invention may comprise, in addition, at least one colouring matter which may be chosen from dyes which are soluble or dispersible in the composition, pigments, pearlescent agents and mixtures thereof. The dyes are preferably fat-soluble dyes, although water-soluble dyes may be used. This colouring matter may represent from 0.001 to 98%, preferably from 0.5 to 85% and even better from 1 to 60% of the total weight of the composition.

For a composition in paste or cast form such as lipsticks or make-up products for the body, from 0.5 to 50% of colouring matter, preferably from 2 to 40% and even better from 5 to 30%, is generally used relative to the total weight of the composition.

The fat-soluble dyes are, for example, Sudan red, D & C Red 17, D & C Green 6, β-carotene, soyabean oil, Sudan brown, D & C Yellow 11, D & C Violet 2, D & C Orange 5, quinoline yellow, annatto They may represent from 0 to 20% of the weight of the composition and even better from 0.1 to 6%. The water-soluble dyes are in particular beet juice, methylene blue and may represent from 0.1 to 6% by weight of the composition (if present).

Preferably, the composition of the invention comprises an advantageously coloured particulate phase which may represent from 0.001 to 50% of the total weight of the composition, preferably from 0.01 to 40% and even better from 0.05 to 30%, and which may comprise pigments and/or pearlescent agents and/or fillers which are normally used in cosmetic compositions.

The expression pigments should be understood to mean inorganicor or organic, White or coloured particles which are insoluble in the liquid fatty phase and which are intended to colour and/or opacify the composition. The expression fillers should be understood to mean lamellar or non-lamellar, mineral or synthetic, colourless or white particles. The expression pearlescent agents should be understood to mean iridescent particles, in particular produced by certain molluscs in their shell or else synthesized. These fillers and pearlescent agents serve in particular to modify the texture of the composition.

The pigments may be present in the composition in an amount of 0.05 to 30% (if present) of the weight of the final composition, and preferably in an amount of 2 to 20%. As inorganic pigments which may be used in the invention, there may be mentioned titanium, zirconium or cerium oxides as well as zinc, iron or chromium oxides and ferric blue. Among the 20′ organic pigments which may be used in the invention, there may be mentioned carbon black, and barium, strontium, calcium (D & C Red No. 7) and aluminium lakes.

The pearlescent agents may be present in the composition in an amount of 0.001 to 20% (if present) of the total weight of the composition, preferably in an amount of the order of 1 to 15%. Among the p arlescent agents which may be used in the inv ntion, there may be mentioned mica coated with titanium oxide, with iron oxide, with a natural pigment or with bismuth oxychloride, such as coloured mica-titanium, gonio-chromatic pigments and, for example, interferential multilayer pigments.

The fillers may be present in an amount of 0.001 to 35% (if present) of the total weight of the composition, preferably 0.5 to 15%. There may be mentioned in particular talc, mica, kaolin, powders of Nylon® (Orgasol in particular) and of polyethylene, polytetrafluoroathylene powders (Teflon®), starch, boron nitride, microspheres of polymers such as Expancel® (Nobel Industrie), Polytrap® (Dow Corning), Polypor® L200 (Chemdal Corporation) and microbeads of silicone resin (Tospearl® from Toshiba, for example), silica.

The composition according to the invention may contain at least one additional nonaqueous compound different from the said aliphatic ester of an ester and from the said oil having a molar mass ranging from 650 to 10000 g/mol, chosen from oils, fatty substances which are pasty at room temperature, waxes, gums, resins, lipophilic polymers and mixtures thereof.

In particular, it contains, in addition, at least one wax. The expression “wax” for the purposes of the present invention is understood to mean a lipophilic fatty compound which is solid at room temperature (25° C.), having a reversible solid/liquid change of state, having a melting point greater than 30° C., which may be up to 200° C., a hardness greater than 0.5 MPa, and exhibiting, in the solid state, an anisotropic crystalline organization. On heating the wax to its melting point, it is possible to make it miscible with oils and to form a microscopically homogeneous mixture, but on bringing the temperature of the mixture back to room temperature, recrystallization of the wax from the oils of the mixture is obtained.

The waxes which may be used in the invention are compounds which are solid at room temperature, intended to structure the composition in particular in the form of a stick; they may be hydrocarbon-based, fluorinated and/or silicone-based and may be of plant, mineral, animal and/or synthetic origin. In particular, they have a melting point greater than 40° C. and even better greater than 45° C.

As wax which may be used in the invention, there may be mentioned those generally used in the cosmetic field: they are in particular of natural origin such as beeswax, Carnauba, Candelilla, Ouricoury, Japan, cork fibre or sugarcane, rice or Montan wax, paraffin, lignite or microcrystalline-waxes, ceresin or ozokerite, hydrogenated oils such as jojoba oil; synthetic waxes such as polyethylene waxes obtained from the polymerization or copolymerization of ethylene and Fischer-Tropsch waxes or alternatively fatty acid esters such as octacosanyl stearate, glycerides which are solid at 40° C. and even better at 45° C., silicone waxes such as alkyl or alkoxy-dimethicones having an alkyl or alkoxy chain of from 10 to 45 carbon atoms, esters of poly(di)methylsiloxane which is solid at 40° C., whose ester chain contains at least 10 carbon atoms; and mixtures thereof.

The gums which may be used in the invention are generally present in solubilized form in an oil, the polymers are solid at room temperature and the resins may be liquid or solid at room temperature.

The nature and the quantity of the gums, pasty substances or waxes depend on the desired mechanical properties and textures. As a guide, the wax may represent from 0.01 to 50%, preferably from 2 to 40% and even better from 5 to 30% of the total weight of the composition.

The additional oils other than the oils having a molar mass ranging from 650 to 10000 g/mol may be hydrocarbon and/or silicone and/or fluorinated oils. These oils may be of animal, plant, mineral or synthetic origin. The expression “hydrocarbon oil” is understood to mean an oil containing mainly carbon and hydrogen atoms and optionally one or more functional groups chosen from hydroxyl, ester, ether and carboxyl functional groups. By way of example of an additional oil which may be used in the invention, there may be mentioned:

hydrocarbon oils of animal origin such as perhydrosqualene;

vegetable hydrocarbon oils such as liquid triglycerides of fatty acids having from 4 to 10 carbon atoms such as triglycerides of heptanoic or octanoic acids or jojoba oil;

linear or branched hydrocarbons of inorganic or synthetic origin such as paraffin oils and derivatives thereof, petroleum jelly;

synthetic esters and ethers in particular of fatty acids such as the oils of formula R₁COOR₂ in which R₁ represents the residue of a higher fatty acid containing from 1 to 40 carbon atoms and R₂ represents a hydrocarbon chain containing from 1 to 40 carbon atoms with 10≦R₁+R₂≦41 such as, for example, isononyl isononanoate, isopropyl myristate, 2-ethylhexyl palpitate, 2-octyldodecyl stearate, 2-octyldodecyl erucate, isostearyl isostearate; hydroxylated esters such as isostearyl lactate, octyl hydroxystearate, octyldodecyl hydroxystearat, diisostearyl malate, heptanoat s, octanoates and decanoates of fatty alcohols; polyol esters such as propylene glycol dioctanoate, neopentyl glycol diheptanoate, diethylene glycol diisononanoate;

fatty alcohols having from 12 to 26 carbon atoms such as octyldodecanol, 2-butyloctanol, 2-hexyldecanol, 2-undecylpentadecanol, oleyl alcohol;

flourinated oils which are optionally partially hydrocarbon-based and/or silicone-based;

silicone oils such as linear or cyclic, volatile or nonvolatile polydimethylsiloxanes (PDMS); polydimethylsiloxanes having alkyl, alkoxy or phenyl groups, pendent or at the end of a silicone chain, groups having from 2 to 24 carbon atoms; phenylated silicones such as phenyl trimethicones (such as the phenyl trimethicone sold under the trade name DC556 by Dow Corning), phenyl dimethicones, phenyl trimethylsiloxy diphenyl siloxanes, diphenyl dimethicones, diphenyl methyldiphenyl trisiloxanes, 2-phenylethyl-trimethylsiloxysilicates;

fatty acids having from 12 to 26 carbon atoms such as oleic acid;

and mixtures thereof.

Additional oils of plant or synthetic origin are preferably used.

The additional oils may represent from 0.001 to 90% of the total weight of the composition, preferably from 0.05 to 60% and even better from 1 to 35%.

The composition of the invention may comprise, in addition, any additional additive normally used in the relevant field, such as water, antioxidants, preservatives, neutralizing agents, lipophilic gelling agents or nonaqueous liquid compounds, aqueous phase gelling agents, dispersants, cosmetic active agents. These additives, with the exception of water which may represent from 0 to 70% and, for example, from 1 to 50% and even better from 1 to 10% of the total weight of the composition, may be present in the composition an an amount of 0.0005 to 20% of the total weight of the composition and even better from 0.001 to 10%.

As cosmetic active agent which may be used in the invention, there may be mentioned vitamins A, E, C, B₃, F, provitamins such as D-panthenol, soothing active agents such as α-bisabolol, Aloe vera, allantoin, plant extracts or essential oils, protective or restructuring agents such as ceramides, “freshening” active agents such as menthol and its derivatives, emollients (cocoa butter, dimethicone), moisturizers (arginine PCA), anti-wrinkle-active agents, essential fatty acids, sunscreens, and mixtures thereof.

Of course, persons skilled in the art will be careful to choose the possible additional additives and/or their quantity such that the advantageous properties of the composition according to the invention are not, or not substantially, impaired by the envisaged addition.

The applications of the compositions according to the invention are many and involve all coloured or noncoloured cosmetic products, and more particularly lipsticks.

The composition of the invention may be provided in the form of a solid composition, compacted or cast in particular as a stick or a dish, a pasty composition or a liquid composition. Advantageously, it is provided in solid form, namely in a hard form (which does not run under its own weight) in particular cast or compacted, for example as a stick or a dish.

It may be provided in the form of a paste, a solid or a cream. It may be an oil-in-water or water-in-oil emulsion, a solid or flexible anhydrous gel or alternatively in the form of a loose or compacted powder and even in biphasic form. Preferably, it is provided in the form of a composition with an oily and in particular anhydrous continuous phase; in this case, it may contain an aqu ous phase in an amount of less than 5%.

The composition according to the invention may be provided in the form of a coloured or noncoloured composition for skin care, in the form of an anti-sun or make-up-removing composition or alternatively in the form of a health care composition. If it contains cosmetic active agents, it may then be used as a nontherapeutic care or treatment base for the skin, for instance the hands or the face, or for the lips (lip balms, protecting the lips from the cold and/or from the sun and/or from the wind), or artificial tanning product for the skin.

The composition of the invention may also be provided in the form of a coloured make-up product for the skin, in particular for the face, such as a blusher, a make-up for the cheeks or an eyeshadow, a make-up product for the body such as a semipermanent tattoo product or a make-up product for the lips such as a lipstick or a lip gloss, optionally having nontherapeutic care or treatment properties, a make-up product for the superficial body growths such as, for example, a nail varnish, a mascara, an eyeliner, a dye or care product for the hair.

Preferably, the composition according to the invention is provided in the form of a lipstick or a lip gloss.

Of course, the composition of the invention should be physiologically acceptable (in particular cosmetically acceptable), that is to say nontoxic and capable of being applied to the skin, the superficial body growths or the lips of human beings.

The expression “cosmetically acceptable” is understood to mean having a pleasant taste, feel, appearance and/or odour, applicable for several days and for several months.

The composition according to the invention may be manufactured by known methods which are generally used in the cosmetic field.

The examples which follow are intended to illustrate, without limitation, the subject of the present invention. The quantities are given as a percentage by mass.

### EXAMPLE 1 AND 2

### Lip Gloss

The compositions presented in the following Table (I) were prepared.

The composition of Example 1 comprises as aliphatic ester of an ester according to the invention, the monoisostearate of hydrogenated castor oil (marketed or manufactured under the reference Salacos HCIS by the company Nisshin Oil Mills) and, as an oil having a molar mass ranging from 650 to 10000 g/mol, the triglyceride of 2-decylt tradecanoic acid (MM=1143.98 g/mol).

The monoisostearate of hydrogenated castor oil was replaced by oxypropylenated lanolin wax in the composition of Example 2 (comparative).

TABLE (I)

| | | Composition (%) | |
|---|---|---|---|
| | | Example 1 | Example 2 |
| | Phase | (invention) | (comparative) |
| Diisostearyl malate | A- | qs | qs |
| | | 100 | 100 |
| Triglyceride of 2-decyl- | | 15.5 | 15.5 |
| tetradecanoic acid | | | |
| Pyrogenic silica | | 4 | 4 |
| (marketed or manufactured | | | |
| under the reference Aerosil | | | |
| R972 by the company Degussa | | | |
| Huls) | | | |
| Titanium dioxide | | 1.8 | 1.8 |
| Red 21 lake | B- | 0.6 | 0.6 |
| Iron oxides | | 0.06 | 0.06 |
| Red 7 lake | | 2.9 | 2.9 |
| Yellow 6 lake | | 3.3 | 3.3 |
| Monoisostearate of | C- | 50 | — |
| hydrogenated castor oil | | | |
| Oxypropylenated lanolin wax | | — | 50 |

Procedure:

A silica gel is prepared by mixing with a turbine mixer the constituents of phase A. The pigments (phase B) are then ground in the silica gel. The mixture is added to phase C in a saucepan. The whole is homogenized using a Raynerie stirrer and heated at 80° C. for one hour, and then poured while hot into kettles.

Sensory Evaluation:

These two compositions were compared by applying as half-lips and evaluated by 5 qualified people according to various criteria.

The composition of Example 1 according to the invention was judged to be more slippery, more glossy, less sticky, and more comfortable to wear over time, than the composition of Example 2.

### EXAMPLES 3 AND 4

### Lip Gloss

The inventors compared the properties of two compositions according to the invention and the prior art. The composition of example 3 comprises the monoisostearate of hydrogenated castor oil (marketed or manufactured under the reference SALACOS HCIS by the company Nisshin Oil Mills) and, as oil having a molar mass ranging from 650 to 10000 g/mol, hydrogenated polybutylene (sold under the trade reference INDOPOL H-100 by the company Amoco and having a molar mass of 965 g/mol.

In the composition of (comparative) Example 4, the monoisostearate of hydrogenated castor oil was replaced with oxypropylenated lanolin wax.

TABLE (II)

| | | Composition (%) | |
|---|---|---|---|
| | | Example 3 | Example 4 |
| | Phase | (invention) | (comparative) |
| Diisostearyl malate | A- | qs | qs |
| | | 100 | 100 |
| Polybutylene | | 5 | 5 |
| Pyrogenic silica | | 4 | 4 |
| (marketed or manufactured | | | |
| under the reference Aerosil | | | |
| R972 by the company Degussa | | | |
| Huls) | | | |
| Titanium dioxide | B- | 1.8 | 1.8 |
| Red 21 lake | | 0.6 | 0.6 |
| Iron oxides | | 0.06 | 0.06 |
| Red 7 lake | | 2.9 | 2.9 |
| Yellow 6 lake | | 3.3 | 3.3 |
| Monoisostearate of | C- | 50 | — |
| hydrogenated castor oil | | | |
| Oxypropylenated lanolin wax | | — | 50 |

The same procedure is used as for Examples 1 and 2 above.

Sensor Evaluation:

These two compositions were compared by applying as half-lips and evaluated by 5 qualified people according to various criteria.

The composition of Example 3 according to the invention was judged to be more slippery, more glossy, less sticky, both during application and over time, than the composition of Example 4.

### EXAMPLES 5 AND 6

### Lip Gloss

The inventors compared the properties of two compositions as presented in Table (III) below. One contains an oil having a molar mass equal to 1143.98 g/mol which is the triglyceride of 2-decyltetradecanoic acid (composition of Example 5) and the other contains the triglyceride of capric/caprylic acids sold under the trade reference ESTOL 3603 MCT OIL by the company UNIQEKA and having a molar mass equal to 494 g/mol (comparative Example 6).

TABLE (III)

| | | Composition (%) | |
|---|---|---|---|
| | | Example 5 | Example 6 |
| | Phase | (invention) | (comparative) |
| Diisostearyl malate | A- | qs | qs |
| | | 100 | 100 |
| Triglyceride of 2-decyl | | 15.5 | — |
| tetradecanoic acid | | | |
| Triglycerides of capric/ | | — | 15.5 |
| caprylic acids | | | |
| Pyrogenic silica | | 4 | 4 |
| (marketed or manufactured | | | |
| under the reference Aerosil | | | |
| R972 by the company Degussa | | | |
| Huls) | | | |
| Titanium dioxide | B- | 1.8 | 1.8 |
| Red 21 lake | | 0.6 | 0.6 |
| Iron oxides | | 0.06 | 0.06 |
| Red 7 lake | | 2.9 | 2.9 |
| Yellow 6 lake | | 3.3 | 3.3 |
| Isostearate of hydrogenated | C- | 50 | 50 |
| castor oil | | | |

The same procedure is used as for the preceding examples.

Sensory Evaluation:

These two compositions were compared by applying as half-lips and evaluated by 5 qualified people according to various criteria.

Composition 5 according to the invention was judged to be smoother, less oily, more pleasant during application and more comfortable and more glossy over time, than the composition of Example 6.

### EXAMPLE 7

Lipstick

| | |
|---|---|
| 2-Octyldodecanol | 16.2% |
| Phenyl trimethicone (sold under the | 4.4% |
| reference DC 556 by Dow Corning | |
| Diisostearyl malate | qs 100% |
| Triglyceride of 2-decyltetradecanoic acid | 2% |
| Tridecyl trimellitate | 10.8% |
| Hydxogenated coconut glycerides (sold | 5% |
| under the trade reference Softisan 100 | |
| by Degussa Huls) | |
| Monostearate of hydrogenated castor oil | 30% |
| (marketed or manufactured under the | |
| reference Salacos HCIS by Nisshin Oil Mills) | |
| Polyethylene wax (MW* = 500) | 8% |
| Microcrystalline wax | 3% |
| Iron oxide | 6% |
| Pearlescent agents | 2.6% |
| BHT | 0.04% |

*MW = w ight-averag molecular mass

Procedure:

The pigment (iron oxide) is ground in the oils (2-octyldodecanol., phenyl trimethicone, diisostearyl malate, triglyceride of 2-decyltetradecanoic acid, tridecyl trimellitate). The mixture is then introduced in a saucepan with the monoisostearate of hydrogenated castor oil and the waxes (hydrogenated coconut glycerides sold under the trade reference Softisan 100 by Degussa Huls, polyethylene wax, microcrystalline wax).

The mixture is then homogenized using a Raynerie stirrer and heated for one hour at 100° C. Then, 5 minutes before casting, the pearlescent agents are incorporated into the mixture. The whole is then cast in a mould at 42° C., then placed for 30 minutes in a refrigerator at −20° C., and then unmoulded.

The stick obtained spreads well, it is smooth, nonsticky, glossy and homogeneous upon application. The formula is also glossy and nonsticky after application, the film obtained is comfortable over time.

1. Cosmetic care or make-up composition for keratin materials comprising a cosmetically acceptable medium containing i) at least one aliphatic ester of an ester resulting from the esterification, by an aliphatic carboxylic acid, of an aliphatic hydroxycarboxylic acid ester and ii) at least one oil of high molar mass ranging from 650 to 10000 g/mol.

2. Composition according to the preceding claim, characterized in that the aliphatic carboxylic acid comprises from 4 to 30 and preferably from 8 to 30 carbon atoms.

3. Composition according to one of the preceding claims, characterized in that the aliphatic carboxylic acid is chosen from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid, docosanoic acid, and mixtures thereof.

4. Composition according to one of the preceding claims, characterized in that the aliphatic carboxylic acid is branched.

5. Composition according to one of the preceding claims, characterized in that the aliphatic carboxylic acid is chosen from isostearic acid, 2-ethylhexanoic acid, hexyldecanoic acid, isoarachidic acid, octyldodecanoic acid, and mixtures thereof.

6. Composition according to one of the preceding claims, characterized in that the aliphatic carboxylic acid is isostearic acid.

7. Composition according to one of the preceding claims, characterized in that the aliphatic hydroxycarboxylic acid ester is derived from a hydroxylated aliphatic carboxylic acid containing from 2 to 40 carbon atoms, preferably from 10 to 34 carbon atoms and even better from 12 to 28 carbon atoms.

8. Composition according to one of the preceding claims, characterized in that the aliphatic hydroxycarboxylic acid ester is derived from a hydroxylated aliphatic carboxylic acid containing from 1 to 20 hydroxyl groups., preferably from 1 to 10 hydroxyl groups and even better from 1 to 6 hydroxyl groups.

9. Composition according to one of the preceding claims, characterized in that the aliphatic hydroxycarboxylic acid ester is chosen from:
a) the partial or total esters of saturated linear monohydroxylated aliphatic monocarboxylic acids;
b) the partial or total esters of unsaturated monohydroxylated aliphatic monocarboxylic acids;
c) the partial or total esters of saturated monohydroxylated aliphatic polycarboxylic acids;
d) the partial or total esters of saturated polyhydroxylated aliphatic polycarboxylic acids;
e) the partial or total esters of aliphatic C₂ to C₁₆ polyols which have reacted with a mono- or polyhydroxylated aliphatic mono- or polycarboxylic acid;
and mixtures thereof.

10. Composition according to one of the preceding claims, characterized in that the aliphatic ester of an ester is chosen from the esters of partial or total esters of aliphatic C₂ to C₁₆ polyols, the said polyols having reacted with a ‘hydroxylated aliphatic’ fatty acid having a saturated or unsaturated chain, containing at least 12′ carbon atoms.

11. Composition according to one of the preceding claims, characterized in that the aliphatic ester of an ester is chosen from the esters of saturated or unsaturated, partial or total esters of aliphatic C₂ to C₁₆ polyols, the said polyols having reacted with ricinoleic acid.

12. Composition according to one of the preceding claims, characterized in that the aliphatic ester of an ester is chosen from the monoisostearate of hydrogenated castor oil, the diisostearate of hydrogenated castor oil, the triisostearate of hydrogenated castor oil and mixtures thereof.

13. Composition according to one of the preceding claims, characterized in that the aliphatic ester of an ester has a melting point ranging from 25 to 60° C., preferably from 30 to 45° C. and/or a hardness ranging from 0.001 to 0.5 MPa, preferably from 0.005 to 0.4 MPa.

14. Composition according to one of the preceding claims, characterized in that the aliphatic ester of an ester represents from 1 to 99%, preferably from 1 to 75% and even better from 5 to 60% of the total weight of the composition.

15. Composition according to one of the preceding claims, characterized in that the oil of high molar mass has a molar mass ranging from 750 to 7500 g/mol.

16. Composition according to one of the preceding claims, characterized in that the oil of high molar mass is chosen from:
lipophilic polymers
linear fatty acid esters having a total number of carbons ranging from 35 to 70
hydroxylated esters,
aromatic esters,
C₂₄-C₂₈ branched fatty acid or fatty alcohol esters,
silicone oils,
oils of plant origin,
and mixtures thereof.

17. Composition according to one of the preceding claims, characterized in that the oil of high molar mass is chosen from polybutylenes, hydrogenated polyisobutylenes, polydecenes, hydrogenated polydecenes, copolymers of vinylpyrrolidone such as PVP/hexadecene, copolymer, pentaerythrityl tetrapelargonate, polyglycery 1-2, triisostearate, tridecyl trimellitate, triisoarachidyl citrate, pentaerythrityl tetraisononanoate, glyceryl triisostearate, glyceryl tri(2-decyltetradecanoate), pentaerythrityl tetraisostearate, polyglyceryl-2 tetraisostearate, pentaerythrityl tetra(2-decyltetradecanoate), phenylated silicones, sesame oil, and mixtures thereof.

18. Composition according to one of the preceding claims, characterized in that the oil of high molar mass represents from 1 to 99%, preferably from 10 to 80% and even better from 5 to 70% of the total weight of the composition.

19. Composition according to one of the preceding claims, characterized in that it is provided in the form of a lipstick or a lip gloss, a blusher or an eyeshadow, a mascara, an ey liner, a nail varnish, an artificial tanning product for the skin, a dye or care product for the hair.

20. Composition according to one of the preceding claims, characterized in that it comprises, in addition, at least one colouring matter.

21. Composition according to the preceding claim, characterized in that the colouring matter is chosen from dyes which are soluble or dispersible in the composition, pigments, pearlescent agents and mixtures thereof.

22. Composition according to claim 20 or 21, characterized in that the colouring matter represents from 0.001 to 98%, preferably from 0.5 to 85% and even better from 1 to 60%, relative to the total weight of the composition.

23. Composition according to one of the preceding claims, characterized in that it contains, in addition, at least one additional nonaqueous compound different from the said aliphatic ester of an ester and from the said oil of high molar mass, chosen from oils, fatty substances which are pasty at room temperature, waxes, gums, resins, lipophilic polymers and mixtures thereof.

24. Composition according to one of the preceding claims, characterized in that it contains, in addition, at least one wax.

25. Composition according to the preceding claim, characterized in that the wax represents from 0.01 to 50%, preferably from 2 to 40% and even better from 5 to 30% of the total weight of the composition.

26. Composition according to one of the preceding claims, characterized in that it comprises, in addition, at least one additional additive chosen from water, antioxidants, preservatives, neutralizing agents, lipophilic gelling agents or nonaqueous liquid compounds, aqueous phase gelling agents, dispersants, cosmetic active agents and mixtures thereof.

27. Composition according to one of the preceding claims, characterized in that it contains at least one cosmetic active agent chosen from vitamins (A, E, C, B₃, F), provitamins, soothing active agents, plant extracts or essential oils, protective or restructuring agents, “freshening” active agents, emollients, moisturizers, anti-wrinkle active agents, essential fatty acids, and mixtures thereof.

28. Composition according to one of the preceding claims, characterized in that it is provided in a cast or compacted form.

29. Composition according to one of the preceding claims, characterized in that it is, provided in the form of a continuous oily phase.

30. Composition according to one of the preceding claims, characterized in that it is provided in an anhydrous form.

31. Composition according to one of the preceding claims, characterized in that it is provided in the form of a lipstick or a lip gloss.

32. Cosmetic method for conferring on a cosmetic composition film properties of slipperiness, of nonstickiness, of gloss and/or of comfort, consisting in introducing into the said composition i) at least one aliphatic ester of an ester resulting from the esterification, by an aliphatic carboxylic acid, of an aliphatic hydroxycarboxylic acid ester and ii) at least one oil of high molar mass ranging from 650 to 10000 g/mol.

33. Method according to claim 32, characterized in that the aliphatic carboxylic acid is in accordance with one of claims 2 to 6.

34. Method according to claim 32 or 33, characterized in that the aliphatic hydroxycarboxylic acid ester is in accordance with one of claims 7 to 9.

35. Method according to one of claims 32 to 34, characterized in that the aliphatic ester of an ester is in accordance with one of claims 10 to 14.

36. Method according to one of claims 32 to 35, characterized in that the oil of high molar mass ranging from 650 to 10000 g/mol is in accordance with one of claims 15 to 18.

37. Use of the combination i) of at least one aliphatic ester of an ester resulting from the esterification, by an aliphatic carboxylic acid, of an aliphatic hydroxycarboxylic acid ester and ii) of at least one oil of high molar mass ranging from 650 to 10000 g/mol, in a physiologically acceptable slippery, nonsticky, glossy and/or comfortable composition.

38. Use of the combination i) of at least one aliphatic ester of an ester resulting from the esterification, by an aliphatic carboxylic acid, of an aliphatic hydroxycarboxylic acid ester and ii) of at least one oil of high molar mass ranging from 650 to 10000 g/mol, in a physiologically acceptable composition, as agent for conferring on the said composition properties of slipperiness, of nonstickiness, of gloss and/or of comfort.

39. Use according to claim 37 or 38, characterized in that the aliphatic carboxylic acid is in accordance with one of claims 2 to 6.

40. Use according to one of claims 37 to 39, characterized in that the aliphatic hydroxycarboxylic acid ester is in accordance with one of claims 7 to 9.

41. Use according to one of claims 37 to 40, characterized in that the aliphatic ester of an ester
